(19) United States
(12) Patent Application Publication
(10) Pub. No.: US 2012/0040390 A1
(43) Pub. Date: Feb. 16, 2012

(54) Method for Introducing Protein and/or Peptide Into Cell

(75) Inventors: Junichiro Futami, Okayama-shi (JP); Hidetaka Nakanishi, Takatsuki-shi (JP); Hidenori Yamada, Okayama-shi (JP); Takashi Kai, Toride-shi (JP)

(73) Assignee: Nippon Shokubai Com, Ltd., Chuo-ku, Osaka-shi (JP)

(21) Appl. No.: 11/597,675

(22) PCT Filed: May 20, 2005
(86) PCT No.: PCT/JP2005/009704

§ 371 Date: Jan. 4, 2008

(30) Foreign Application Priority Data
May 21, 2004 (JP) 2004-151853

(51) Int. Cl. C12Q 1/02; C12N 5/07

(52) U.S. Cl. 435/29; 435/325

(57) ABSTRACT

The present invention provides a method of intracellular introduction by which a protein and/or peptide can be efficiently introduced into living cells and which makes it possible to effectively analyze the behavior and function of the protein and/or peptide introduced and can be suitably utilized/applied, for example in the technology “live cell imaging” which realizes the immunohistochemical visualization in living cells under physiological conditions.

The method of intracellular introduction is a method for introducing a protein and/or peptide into a cell, which comprises the process of introducing a conjugate generated by binding of a compound adsorbable on a cell surface to a protein and/or peptide via a polysulfide bond into a cell and treating with a reducing agent.

### TECHNICAL FIELD

The present invention relates to a method of introducing a protein and/or peptide into a cell. More particularly, it relates to a protein and/or peptide conjugate as well as a method of efficiently introducing a protein and/or peptide into a cell using such conjugate and an improvement in such method.

### BACKGROUND ART

In the field of immunohistochemistry, a technique has so far been used which comprises introducing a protein or antibody labeled with a fluorescent probe, for instance, and capable of recognizing a specific molecule (conjugate of a labeled protein with a cationic compound) into dead cells whose cell membrane shows improved permeability as a result of immobilization and then removing the nonspecifically bound fraction under appropriate rinsing conditions to visualize only the specifically bound protein in the cells. However, the results of analysis of the behavior of an immobilized protein in cells are not always in agreement with the phenomenon actually occurring while the cells retain their original functions. In view of this and, further, in view of the increased demand for behavioral observation in living cells, it is strongly demanded that a technique be developed of the technology “live cell imaging”, which realizes the immunohistochemical visualization under physiological conditions in living cells.

As for the method of introducing a protein itself into cells, use has been made, in view of the necessity of cell membrane permeation, of such a special technique as microinjection, or the technique comprising encapsulating a protein within liposomes or like capsular bodies and contacting them with the cell membrane to thereby causing intracellular introduction of the contents (protein etc.) by fusion or by uptake through the endocytosis owing to cellular phagocytosis. A method of intracellular introduction which utilizes the receptor-mediated pathway with one of various receptors expressed on the cell surface being set as a target and the ligand as a carrier has also been under investigation although the cell species applicable are restricted.

In regard to the method of introducing a protein into living cells, the Applicant has already developed a method of efficiently introducing a protein into living cells which comprises utilizing the electrostatic adsorption of a protein cationized in advance on the live cell surface charged negatively. This technique, which is disclosed in “METHOD FOR INTRODUCING PROTEIN OR PEPTIDE INTO CELL” (Japanese Kokai Publication No. 2004-049214), has established a technology of introducing various proteins prepared extracellularly in advance into living cells in a simple and easy manner. However, for example when it is applied to “live cell imaging” for observing the behavior or localization of a fluorescence-labeled protein introduced into cells under a fluorescence microscope, the fluorescence from the protein merely adhering to the cell surface or vicinity thereof and/or the granular fluorescence possibly resulting from the retention, in endosomes and/or lysosomes, of the protein taken up into cells by endocytosis (phagocytosis) may form a background (background light) and, therefore, there is room for improvement so that the exact behavior of the protein in living cells may be observed in more detail.

By the way, with regard to the technology of intracellular introduction of an active biopolymer from the endosome into the cytoplasm, “Biochemica et Biophysica Acta”, 2003, Volume 1613, pages 28 to 38 (written by Marie-Andrée Yessine etc., published by Elsevier Science) discloses that a polymer sensitive to pH changes is effective in disrupting the endosome. However, in this technology, there is room for contrivance for sufficiently eliminating the background due to the protein and/or peptide retained in the endosome or lysosome and thereby making it possible to more accurately analyze the behavior and function of the protein and/or peptide in living cells.

In the case of gene transfer, labeling of the gene with a fluorescent substance, for instance, may possibly influence the transcriptional function and, therefore, the gene transfer is generally carried out without labeling; thus, the background problem resulting from the retention within the endosome may be said to be a protein- and/or peptide-specific phenomenon.

### DISCLOSURE OF INVENTION

The present invention, which has been made in view of the above-discussed state of the art, has for its object to provide a method of intracellular introduction by which a protein and/or peptide can be efficiently introduced into living cells and which makes it possible to effectively analyze the behavior and function of the protein and/or peptide introduced and can be suitably utilized/applied, for example in the technology “live cell imaging” which realizes the immunohistochemical visualization in living cells under physiological conditions.

The present inventors had already found that a method of efficiently introducing a protein into living cells which comprises utilizing the electrostatic adsorption of a protein cationized in advance on the living cell surface charged negatively (Japanese Patent Application No. 2002-287280). However, there is room for contrivance so that the exact behavior of the protein in living cells may be observed in more detail.

And, the present inventors, who had already found, with regard to the method of intracellular introduction of a protein and/or peptide, that a conjugate derived from a protein and/or peptide and a compound adsorbable on a cell surface is efficiently incorporated into a living cell (or cells) and such a technology of intracellular introduction is therefore useful. They made further various investigations concerning such method of intracellular introduction and found that when a polysulfide bond, such as a disulfide bond, is disposed between the protein and/or peptide and the compound adsorbable on a cell surface, which form the above-mentioned conjugate, so that the polysulfide bond may be exposed to a solvent, the resulting conjugate is introduced into cells and then the cell surface is treated with a reducing agent, the exposed polysulfide bond is rapidly reduced because of the ready cleavability (cleaving (cutting)-ability) of the polysulfide bond, and then the protein and/or peptide and the compound adsorbable on a cell surface are dissociated from each other. They found that that portion of the protein and/or peptide adsorbed on the cell surface via the compound adsorbable on a cell surface loses the ability to be adsorbed on the cell surface after dissociation of the both from each other and, as a result, that portion of the protein and/or peptide adsorbed on the cell surface can be satisfactorily removed by rinsing and found that the function or functions of the protein and/or peptide introduced into cells can thus be effectively analyzed; they came to realize that the above object can be successfully accomplished in the manner mentioned above.

Such method of intracellular introduction according to the present invention is a method which can realize highly precise function analysis of proteins and/or peptides and is adequately utilizable or applicable in the fields of drugs (medicines) and research reagents in which proteins and/or peptides are used. The method is particularly suitable for use in analyzing the behavior and/or localization of a labeled protein and/or peptide introduced into cells. On the occasion of observing a labeled protein and/or peptide under a fluorescence microscope, that portion of the labeled protein and/or peptide adsorbed on the cell surface is generally observed as a background (background light). However, when a reducing agent is added thereto and allowed to react, the polysulfide bond between the protein and/or peptide and the compound adsorbable on a cell surface, which remain merely adsorbed on the cell surface without incorporation into cells, is reduced and that portion of the protein and/or peptide leaves the cell surface, so that it becomes possible, on the occasion of observation, to observe an image with a marked reduction in background level.

Further, regarding the function analysis of a labeled protein and/or peptide introduced into cells, the inventors, supposing that the conjugate between a protein and/or peptide and a compound adsorbable on a cell surface be incorporated into cells via such a pathway as endocytosis, paid their attention to the possibility of retention of the conjugate in endosomes. And, they found that when a technology of disrupting endosomes in living cells is utilized, it becomes possible to release the conjugate retained in endosomes into the cytoplasm and, therefore, even when a labeled protein and/or peptide is used in the conjugate, the conjugate can be satisfactorily released into the cytoplasm. When the fluorescence of a labeled protein and/or peptide is observed, the fluorescence from the conjugate retained in endosomes is generally observed as a background. The inventors found that when the endosomes in living cells are disrupted, for example, by adding a polymer whose solubility changes in a pH-dependent manner, the inclusion in the endosomes is released into the cytoplasm and it becomes possible to observe an image reduced in background level. Such and other findings have now led to completion of the present invention.

The method of intracellular introduction according to the present invention can be utilized in a wide range, from immunohistochemical analysis, that has so far been carried out using immobilized cells, to dynamic visualization of molecules within living cells (“live cell imaging”). In particular, it constitutes a technology suitably utilizable or applicable in the technology of image analysis.

That is to say, the present invention is a method for introducing a protein and/or peptide into a cell,

which comprises the process of introducing a conjugate generated by binding of a compound adsorbable on a cell surface to a protein and/or peptide via a polysulfide bond into a cell, and treating with a reducing agent.

The present invention is also a method for introducing a protein and/or peptide into a cell,

wherein the protein and/or peptide is a labeled one,

the method comprising the process of adding an endosome-disrupting substance in a living cell to thereby disrupt the endosome.

In the following, the present invention is described in detail.

The method of intracellular introduction according to the present invention may be either (A) a method comprising the process of introducing a conjugate generated by binding (coupling) a compound adsorbable on a cell surface to a protein and/or peptide via a polysulfide bond into a cell, and treating with a reducing agent or (B) a method of introducing a labeled protein and/or peptide into a cell which comprises the process of adding an endosome-disrupting substance (a substance capable of disrupting an endosome) in a living cell to thereby disrupting the endosome, or a combination of these methods (A) and (B).

The above-mentioned method of intracellular introduction is preferably an extracellular (in vitro) method of intracellular introduction. Thus, it is preferably a method of introducing a protein and/or peptide in vitro into a cell collected from within a living body or the like. In this case, the processes of conjugate introduction, reduction treatment with a reducing agent, endosome disruption, etc., which are to be mentioned later herein, are carried out in vitro.

The above-mentioned method (A) of intracellular introduction comprises the processes of introducing a specific conjugate into cells and treating with a reducing agent and, as for the method of conjugate introduction into cells, the introduction can be realized, for example, by adding the conjugate of the present invention or a solution containing the conjugate to a medium containing cells into which the protein and/or peptide is to be introduced and, then, cultivating the cells under adequate cultivating conditions including an appropriate cultivating temperature and an appropriate cultivation time. In this manner, the conjugate of the present invention is incorporated into cells, and the amount of the conjugate incorporated into cells increases with the lapse of time. Thus, when a cationic compound (compound having the cationic character) is used as the compound adsorbable on a cell surface, the conjugate is presumably incorporated into cells by a mechanism due to the electrostatic interaction between the positive charge which the conjugate has and the negative charge on the cell surface, since the cell surface is constituted of anionic molecules, typically heparin sulfate proteoglycans and thus is negatively charged. Presumable as the electrostatic interaction-due mechanism are, for example, the endocytosis-due mechanism and the mechanism involving direct permeation through the cell membrane.

In cases where intracellular conjugate introduction is carried out in that manner, the conjugate partly occurs in a state merely adhering to the cell surface without incorporation into cells. By carrying out further treatment with a reducing agent according to the present invention, it becomes possible to release the protein and/or peptide from the conjugate adhering to the cell surface and thereby effectively carry out the function analysis, under physiological conditions, of the protein and/or peptide introduced into cells.

The “treating (treatment) with a reducing agent” so referred to herein means the reduction of the polysulfide bond of the conjugate of the present invention using a reducing agent. In accordance with the present invention, a polysulfide bond (such as a disulfide bond) is disposed, in a solvent-exposed state, between the protein and/or peptide and the compound adsorbable on a cell surface, so that it becomes possible to carry out the reduction treatment under mild conditions. As a result of such reduction treatment, the protein and/or peptide and the compound adsorbable on a cell surface dissociate from each other and, as a result of the dissociation of both results, the protein and/or peptide adsorbed on the cell surface via the compound adsorbable on a cell surface loses its ability to bind to the cell surface, so that the protein and/or peptide can be sufficiently rinsed away/removed from the cell surface. As a result, for example when a labeled protein and/or peptide is observed under a fluorescence microscope, it becomes possible to observe an image while the background (background light) resulting from the adsorption, on the cell surface by an electrostatic interaction, of the conjugate of the labeled protein and/or peptide with the compound adsorbable on a cell surface is satisfactorily reduced.

In this manner, the intracellular introduction of the present invention also constitutes a method of rinsing a protein and/or peptide from a cell surface and further constitutes a method of rinsing away/removing or eradicating a signal due to a conjugate of a protein and/or peptide and a compound adsorbable on a cell surface as remaining adsorbed on a cell surface. Furthermore, it serves also as a method especially preferably used in image analysis of proteins and/or peptides.

Under neutral or weakly alkaline conditions, the solvent-exposed disulfide bond undergoes the reaction according to the formula (1) given below via the disulfide exchange reaction caused by the reducing agent occurring in excess.

Carrier-SS-Probe+excess R—SH [symbol] Carrier-SH+Probe-SH+excess R—SH+R—SS—R+Carrier-SS—R+Probe-SS—R (1)

Here, when dithiothreitol (DTT) is used in slight excess as the reducing agent, the reaction represented by the following formula (2) proceeds.

Carrier-SS-Probe+DTT [symbol] Carrier-SH+Probe-SH+Oxidized DTT (2)

In the above reaction formulas (1) and (2), “Carrier” means a carrier for intracellular introduction, “SS” represents a disulfide bond, “Probe” means a protein and/or peptide labeled with a fluorescent probe, “R” represents an arbitrary atom or atomic group, “SH” means a thiol group (mercapto group), and “DTT” represents dithiothreitol (HS—CH₂(CHOH)₂CH₂—SH).

In the reaction systems shown by the above formulas (1) and (2), a variety of mixed disulfides are formed in (1) while, in (2), DTT causes the transition, of the disulfide bond in the reaction system to a disulfide bond in the DTT molecule. Therefore, DTT is used as a particularly preferred reducing agent in the practice of the present invention.

On the other hand, the conjugate introduced into cells, under the reductive environment in the cytoplasm, undergoes the reaction shown by the above formula (1) mainly under the action of reduced-form glutathione etc., hence is expected to be dissociated into the conjugate constituents, namely the protein and/or peptide and the compound adsorbable on a cell surface. Therefore, on the occasion of the binding of the protein and/or peptide to an intracellular target site as well, the protein and/or peptide can perform its original function without being influenced by the compound adsorbable on a cell surface. In view of these advantages, it is desirable that a readily cleavable site such as a disulfide bond be disposed between the protein and/or peptide and the compound adsorbable on a cell surface.

As regards the action mechanism of the present invention, the outline of the method of background reduction in intracellular protein (and/or peptide) visualization is schematically shown in FIG. 1 taking, as an example, the case in which a protein is used as the introduction-aimed protein and/or peptide and a cationic polymer or a disulfide bond (SS)-containing cationic carrier (compound resulting from coupling of a cationic polymer-avidin conjugate to biotin) is used as the compound adsorbable on a cell surface. However, the present invention is never limited to the mode of embodiment shown in FIG. 1.

The above-mentioned method of intracellular introduction may also comprise further adding an endosome-disrupting substance after the above-mentioned reduction treatment for endosome-disrupting treatment. Namely, the conjugate of the present invention is presumably incorporated into the cell inside via such a route as endocytosis and, since the conjugate is retained in endosomes, it becomes possible to release the endosome contents into the cytoplasm by disrupting the endosomes. This makes it possible to introduce the protein and/or peptide into the intracellular target site to amore satisfactory extent. Further, for example when a labeled protein and/or peptide is used as the introduction-aimed substance, the fluorescent probe from the conjugate retained in endosomes may be observed as a background in some cases. By disrupting the endosomes in living cells to release the endosome contents into the cytoplasm, it becomes possible to observe an image with a satisfactory reduction in background.

The endosome-disrupting substance mentioned above is not particularly restricted but preferably is a substance capable of disrupting the endosome membrane by changing the form or shape thereof in response to the changes in surrounding environment within the endosomes, for example in such an external stimulus and/or substance as temperature, pH, salt concentration and/or light energy. Such substance is not particularly restricted but preferably is a polymer. Thus, for example, temperature-sensitive polymers, polymers whose solubility is dependent on the pH and/or salt concentration, and polymers having a functional dye moiety can be suitably used. For example, the method of disrupting endosomes which comprises using a substance capable of being insolubilized at the stage of acidification within the late-stage endosome vacuoles is preferably utilized. The principle of the action is not clear but the endosome membrane is presumably made unstable by the substance insolubilized and precipitated as the endosomes are acidified, with the result that the conjugate of the present invention is released. Also employable as the method of disrupting endosomes are the methods causing morphological changes by means of external stimuli such as ultrasonic waves, light, temperature, etc.

The above-mentioned substance insolubilized under acidic conditions is preferably a polymer whose solubility changes depending on the pH. The polymer whose solubility changes depending on the pH is a polymer whose solubility changes depending on the pH of the solution containing the same and preferably is a polymer which is completely soluble in an alkaline aqueous solution and whose solubility decreases in a neutral or acidic solution. Suited for use as such a polymer is a hydrophobic group-containing and carboxyl group-containing polymer.

As the hydrophobic group- and carboxyl group-containing polymer, there may be mentioned, for example, synthetic polymers such as acrylic polymer, urethane polymer, polyester and polyamide, and modification of such polymer and, further, natural polymer such as macromolecular polysaccharide, natural rubber, polypeptide and nucleic acid, and modification thereof. The polymer-constituting monomer may have a hydrophobic group and/or a carboxyl group beforehand or, alternatively, a hydrophobic group and/or a carboxyl group may be bound to the polymer. As the hydrophobic group, there may be mentioned an alkyl group and an aromatic ring-containing group.

As suitable examples of such hydrophobic group- and carboxyl group-containing polymers, there may be mentioned polymers prepared by polymerization and purification in the conventional manner from a carboxyl group-containing monomer such as (meth) acrylic acid, cinnamic acid, crotonic acid, maleic acid, itaconic acid or fumaric acid, and a (meth)acrylate ester such as methyl(meth)acrylate; an unsaturated aromatic such as styrene or alphamethylstyrene.

In the following, compounds and so forth that can be used in the practice of the present invention are described in detail.

The protein and/or peptide in the present invention means a compound produced by binding two or more amino acids with each other via a peptide bond. Usable as the protein and/or peptide in the present invention is, for example, a peptide, an enzyme, an antibody, or a protein and/or peptide which has other functions (physiological activity such as pharmacological action) and is effective as a medicine or a drug. The molecular weight is preferably within a range of from 100 to 1000000. Among them, a protein and/or peptide having a thiol group (SH-group) is preferable, and a protein and/or peptide having a cysteine (Cys) residue is more preferable. Incidentally, the protein in the present invention includes a conjugated protein formed by conjugation of a sugar chain, lipid and/or a phosphate group to the protein. Further, the structure of the protein may either be a native state or a denatured state.

The conjugate in accordance with the present invention is the product of binding, via a polysulfide bond, of a compound adsorbable on a cell surface to a protein and/or peptide. The polysulfide bond includes, within the meaning thereof, bonds formed between or among two or more sulfur atoms, for example a disulfide bond (S—S bond), a trisulfide bond (S—S—S bond). Among them, a product of coupling via a disulfide bond is preferred. The protein and/or peptide and the compound adsorbable on a cell surface may be bound to the polysulfide bond either directly without any intermediary or via a spacer resulting from the use of, for example, a divalent crosslinking reagent known in the art. In the practice of the present invention, the use of a disulfide bond-containing crosslinking reagent at the spacer site is particularly preferred. In cases where the protein and/or peptide and the compound adsorbable on a cell surface are bound together via a disulfide bond, a disulfide bond-containing covalent bond can be formed between the cysteine residue thiol group in the protein and/or peptide molecule and the compound adsorbable on a cell surface using one or two or more of such reagents as N-succinimidyl 3-(2-pyridyldithio) propionate (SPDP), 4-succinimidyloxycarbonyl-α-methyl-α-(2-pyridyldithio)toluene (SMPT), and derivatives of these. The above-mentioned reagents each may be in the form of a powder or a solution in a medium. The amount of each reagent is not particularly restricted but is to be established so as to be appropriate for coupling the compound adsorbable on a cell surface to the protein and/or peptide.

An example in which a disulfide bond is formed using PEI (polyethylenimine) as the compound adsorbable on a cell surface and SPDP at the spacer site is schematically shown below in terms of the formula (3).

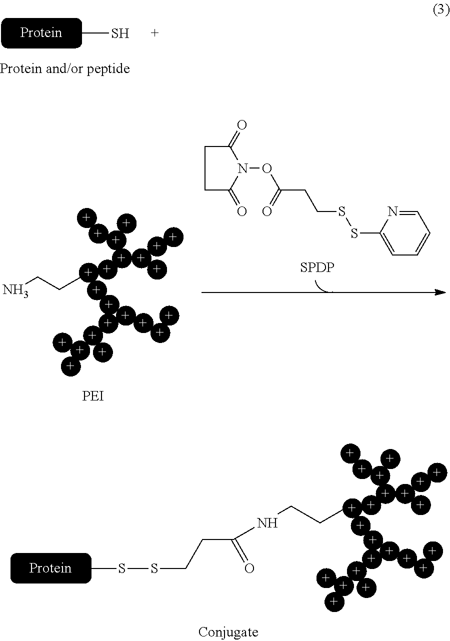

The above-mentioned compound adsorbable on a cell surface, which is a constituent of the above-mentioned conjugate, may be any compound adsorbable on a cell surface, for example on the cell membrane, and a cationic compound such as a cationic group-containing polymer and a cationized complex are preferably used. Lipid may also be used.

The cationic group-containing polymer to be used in the present invention may be any of the polymers having an atom capable of occurring as a cation in an aqueous solution, for example, a polymer having a cation value of more than 2 and not more than 30000. When the cation value is not more than 2, it may not introduce sufficient positive electric charge into the protein and/or peptide. When it exceeds 30000, the effects of the present invention may possibly be not fully produced. More preferably, it is not more than 20000, still more preferably not more than 2500, especially preferably not more than 250. Most preferably, it is not less than 4 and not more than 70.

The above-mentioned cation value is defined as a value obtained by dividing the product of the amine value of a polymer (mmol/g) and the number average molecular weight of the polymer by 1000, and the amine value is an indicator of the total amine in a sample compound and it is represented as mmol number of amines that exist in 1 g of the sample compound. The amine value of the sample compound may be measured according to a common method for quantitative analysis of amino groups. As such common quantitative analysis of amino groups, it can be used a method described in “Shin-jikkenn Kagaku Koza Vol. 13 Yuki-Kagaku-Kozo I” (Chemical Society of Japan, MARUZEN Co., Ltd., Nov. 20, 1978, pp. 88-99) and Colloidal titration (R. Senju, “Colloidal titration method”, 1st Ed., Nankodo Co., Ltd., Nov. 20, 1969). A suitable quantitative method should be chosen for accurate measurement of the amine value, in consideration of a form and solubility of the sample compound, and contaminants in the sample. The amine value of the above-mentioned polymer is preferably not less than 1 but not more than 30, more preferably not less than 5 but not more than 25.

The number average molecular weight of the above-mentioned cationic group-containing polymer is preferably not less than 100 but not more than 100000 in consideration of efficiency for intracellular introduction and handling property. More preferably, it is not less than 100 but not more than 10000, still more preferably not less than 200 but not more than 3000. In addition, the number average molecular weight of the polymer is preferably measured by gel permeation chromatography (GPC).

Suited for use as the above-mentioned cationic group-containing polymer are, for example, a polymer (copolymer) having one single species or a combination of two or more species of a polyalkylenepolyamine skeleton, a polyallylamine skeleton, a polyvinylamine skeleton, a poly(dialkylaminoalkyl (meth)acrylate) skeleton, a poly(meth)acrylic dialkylaminoalkylamide skeleton, a polyamidine skeleton, a polyvinylpyridine skeleton or polyvinylimidazole skeleton, and a copolymer thereof. Further, a salt of these polymers e.g. primary, secondary, tertiary, or quarternary ammonium salt are preferable. Additionally, those polymers, which are chemically altered or modified, may be used.

The above-mentioned cationic group-containing polymer are, for example, polyalkylenepolyamine such as polyalkylenimine (e.g. polyethylenimine, polypropylenimine); polyallylamine such as polyallylamine and polydiallyldimethylammonium chloride; polyvinylamine such as Hofmann decomposition product of polyacrylamide, hydrolysate of polyvinylacetamide, a hydrolysate of polyvinylphthalimide and a hydrolysate of N-vinylformamide polymer; dialkylaminoalkyl(meth)acrylamide (co)polymer such as dimethylaminopropyl(meth) acrylamide (co)polymer; dialkylaminoalkyl(meth)acrylate (co)polymer such as polymethacryloyloxyethyl trimethylammonium chloride; polyamidine; polyvinylpyridine; polyvinylimidazole; a dicyandiamide condensate; epichlorohydrin-dialkylamine condensate such as an epichlorohydrin-dimethylamine condensate; dialkylamine-alkyldihalide condensate such as a dimethylamine-ethylenedichloride condensate; polyvinylimidazoline; polyvinylbenzyl trimethylammonium chloride; carboxy methyl cellulose quarternary ammonium (quarternary ammonium CMC); glycolchitosan; cationized starch and the like. Among these, polyethylenimine (PEI) is preferable.

Following is the theoretical amine values of typical polymers among the above-mentioned cationic group-containing polymers.

TABLE 1

| | Amine |
|---|---|
| | value |
| | (mmol/g)* |
| polyethyleneimine | 23 |
| polyvinylamine | 23 |
| polyallylamine | 17 |
| polydiallyldimethylammonium chloride | 6.2 |
| polymethacryloyloxyethyl trimethylammonium chloride | 4.8 |
| polymethacryloylaminopropyl trimethylammonium chloride | 4.5 |
| polyamidine | 6.0 |
| polyvinylbenzyl trimethylammonium chloride | 6.3 |
| polyvinylpyridine | 10 |
| polyvinylimidazole | 11 |
| epichlorohydrin-dimethylamine condensate | 7.2 |
| dimethylamine-ethylenedichloride condensate | 9.3 |

*theoretically maximum value

In the above table, the theoretical amine values are calculated by multiplying a reciprocal of molecular weight of a component monomer of the polymer by 1000. The amine values experimentally determined by the above-mentioned method are almost identical with the theoretical value within a range of measurement errors. The cation value of the polymer may be calculated on the basis of the amine value determined by the above-mentioned method. The amine value may be optionally varied by changing a synthesizing method of the polymer, copolymerizing with other components, or chemical modification of the polymer.

Referring to the compound adsorbable on a cell surface, the cationized complex is preferably a complex resulting from binding of the cationic group-containing polymer mentioned above to a protein and/or peptide and, for example, use is preferably made of (I) a complex resulting from binding of a cationic group-containing polymer to avidin, which is one of those complex-forming proteins and/or peptides; (II) a complex resulting from binding of a cationic group-containing polymer to protein A and/or protein G, which is one of those complex-forming proteins and/or peptides; or (III) a complex resulting from binding of a cationic group-containing polymer to an antibody, which is one of those complex-forming proteins and/or peptides. In the method for forming those complex, one or two or more of such reagents as dehydration/condensation agents such as 1-ethyl-3-(3-dimethylaminopropyl)-carbodiimide hydrochloride (EDC), N,N-dicyclohexylcarbodiimide (DCC); N-succinimidyl 3-(2-pyridyldithio)propionate (SPDP); 2-iminothiolane; N-(4-maleimidebutyryloxy)succinimide (GMBS) are preferably used. The amounts of these reagents are not particularly restricted but are to be established so as to be appropriate for binding the cationic group-containing polymer to the protein and/or peptide.

In cases where the above-mentioned complex (I) is used, the introduction-aimed protein and/or peptide is preferably biotinylated in advance using a polysulfide bond-forming reagent, such as the above-mentioned SPDP or SMPT, so that it may be bound to avidin in the complex to form a conjugate resulting from binding of the compound adsorbable on a cell surface to the protein and/or peptide via a polysulfide bond. The above-mentioned avidin, protein A and protein G include not only their naturally occurring forms but also forms genetically engineered for the purpose of improving their function.

The above-mentioned conjugate in the present invention, if necessary, may be labeled. The labeling method is not particularly restricted as long as it is a common method, but preferably is a method using such as fluorescent labeling, autoradiography, high electron-dense material, and insoluble pigment producing enzyme. Especially preferred is a method comprising labeling the conjugate by covalently binding the conjugate with the fluorescent labeling compound. In the present invention, a conjugate generated by binding of a compound adsorbable on a cell surface to a labeled protein and/or peptide is preferably used, and thereby the effects of the present invention may be fully brought out.

A fluorescent substance to be used for the fluorescent labeling is not particularly restricted and, for example, the compound having a fluorescent group such as pyrene, an anthraniloyl group, a dansyl group, fluorescein, rhodamine, or nitrobenzoxadiazol. The compound having the above fluorescent groups is disclosed in the literatures (refer to e.g., Hiratsuka Toshiaki, “Tanpakushitu, Kakusan, Koso”, 1997, Vol. 42, No. 7), and the compound can be introduced into a protein and/or peptide and the like by conventional methods.

In the above-mentioned reduction treatment process, the reducing agent means a compound capable of reducing the polysulfide bond, such as disulfide bond, to a thiol group, and a thiol group-compound is preferable used. More preferred is a water-soluble compound and, for example, DTT or β-mercaptoethanol is preferably used. Most preferred is DTT.

In carrying out the intracellular introduction method of the present invention, it is also possible to use a test kit for intracellular introduction of a protein and/or peptide which comprises a container containing a compound adsorbable on a cell surface (such container hereinafter referred to also as “container 1”), a container containing a reagent for coupling (binding) the compound adsorbable on a cell surface to a protein and/or peptide via a polysulfide bond (such container hereinafter referred to also as “container 2”), and a container containing a reducing agent (such container hereinafter referred to also as “container 3”). By using such a test kit, it becomes possible to carry out the intracellular introduction and reduction treatment of the conjugate resulting from binding of the compound adsorbable on a cell surface to the protein and/or peptide in an efficient and simple manner.

The method of using the above test kit is not particularly restricted provided that the effects of the present invention can be produced. Preferred are, for example, the method comprising adding the introduction-aimed protein and/or peptide to the container 1, then adding the contents of the container 1 to the container 2, adding the contents thereof to a medium containing cells into which the protein and/or peptide is to be introduced and, after any of various cultivations etc., adding thereto the contents of the container 3; and the method comprising adding the contents of the container 1 to the container 2, then adding the introduction-aimed protein and/or peptide to the container 2, adding the contents thereof to a medium containing cells into which the protein and/or peptide is to be introduced and, after any of various cultivations etc., adding thereto the contents of the container 3.

Also usable as the intracellular introduction test kit is a test kit comprising a container containing a conjugate resulting from binding of a compound adsorbable on a cell surface to a protein and/or peptide via a polysulfide bond and a container containing a reducing agent.

The container used in the above-mentioned test kit is preferably a container having a capacity of 0.5 to 10 ml. The container may be sterilized by autoclaving, ultraviolet irradiation, or gamma beam irradiation, for instance. The container inside may be coated for preventing mold growth and/or putrefaction of the substance within the container. Further, it is also preferred that the contents be charged into containers under an inert gas such as nitrogen or argon. After charging the above-mentioned substances into the respective containers, the containers are preferably stored in a tightly closed condition at low temperatures not exceeding 25° C.

Suitable as the mode of introducing a protein and/or peptide into cells by the above-mentioned intracellular introduction method (B) is the mode in which a conjugate resulting from binding of a compound adsorbable on a cell surface to the introduction-aimed protein and/or peptide is formed and the conjugate is introduced into cells. The bond between the protein and/or peptide and the compound adsorbable on a cell surface is not particularly restricted but may be any chemical bond. Preferably, it is a covalent bond. More preferably, it is a polysulfide bond, still more preferably a disulfide bond, among others. The protein and/or peptide and the compound adsorbable on a cell surface may be directly bound together without any intermediary between them, or may be bound together via a spacer using a divalent crosslinking reagent known in the art. In the practice of the present invention, the use of a disulfide bond-containing crosslinking reagent at the spacer site is particularly preferred.

The method of forming the conjugate resulting from binding of the protein and/or peptide and the compound adsorbable on a cell surface via a polysulfide bond, the compound and reagent to be used, the method of labeling, and the method of introducing the conjugate into cells, etc. are as described hereinabove. The substance for disrupting an endosome in a living cell and the method of disrupting the endosome are also as described hereinabove. Preferably utilized among others, however, is the method which comprises using a substance insolubilized at the stage of acidification within the late-stage endosome vacuoles to disrupt the endosomes.

In carrying out the above-mentioned method of disrupting endosomes, it is also preferable to use a test kit for intracellular introduction of a protein and/or peptide which comprises a container containing an endosome-disrupting substance (such container herein referred to also as “container 4”). By this, the protein and/or peptide can be introduced into cells in an efficient and simple manner via the process of endosome disruption. The method of using such test kit is not particularly restricted provided that the effects of the present invention can be produced. Preferred is, for example, the method comprising adding the introduction-aimed protein and/or peptide to the container 1 mentioned above, then adding the contents of the container 1 to the container 2, adding the contents thereof to a medium containing cells into which the contents thereof are to be introduced and, after any of various cultivations etc., adding thereto the contents of the container 4.

The present invention is also concerned with the product obtained by the above-mentioned intracellular introduction method (A) and/or (B). Thus, (A) the product obtained by introducing a conjugate resulting from binding of a compound adsorbable on a cell surface to a protein and/or peptide via a polysulfide bond into cells, and treating with a reducing agent and (B) the product obtained by introducing a labeled protein and/or peptide (preferably a labeled derivative of a conjugate resulting from binding of the introduction-aimed protein and/or peptide to a compound adsorbable on a cell surface) into cells and then adding a substance capable of disrupting an endosome in a living cell to thereby disrupt the endosome also constitute an aspect of the present invention.

Such products are judiciously used, for example, reagents not only in the “live cell imaging” technology but also as drugs (medicines) or reagents, in supporting new drug development or in regenerative medicine, among others.

The intracellular introduction method of the present invention, which has the constitution described hereinabove, by which a protein and/or peptide can be efficiently introduced into living cells and which makes it possible to effectively analyze the behavior and function of the protein and/or peptide introduced and can be suitably utilized/applied, for example in the technology “live cell imaging” which realizes the immunohistochemical visualization in living cells under physiological conditions. Further, it is a technology that may be applied to medicines, reagents, support for drug development, and regenerative medicine.

### BRIEF DESCRIPTION OF DRAWINGS

FIG. 1 is a schematic drawing illustrating the outline of the method of reducing the background in intracellular protein visualization in the intracellular introduction method of the present invention.

FIG. 2 is a drawing schematically illustrating the binding mode of eGFP-NLS-SS-Biotin+PEI600-Avidin in Example 1.

FIG. 3-A is a representation of the results of observation in Example 1 confirming that the eGFP-due green fluorescence (Probe) and the red fluorescence of RITC-labeled PEI-Avidin (Carrier) dissociated in cells and the Probe was localized in the intracellular nucleus and the vicinity thereof.

FIG. 3-B is a representation of the results of observation, in Example 1, of the appearance of the cell surface and of the cell inside upon total visualization of the Probe-due fluorescence, by means of fluorescent probe and transmitted light.

FIG. 3-C is a representation of the results of observation, in Example 1, of the appearance of the cell surface and of the cell inside after carrying out the intracellular introduction method of the present invention, upon total visualization of the Probe-due fluorescence, by means of fluorescent probe and transmitted light.

FIG. 3-D is a representation of the observation results obtained by enlarging a part of the image observed in FIG. 3-C and further analyzing the localization of intracellular fluorescence in the direction of the Z-axis.

FIG. 4-A is a representation of the results of observation, in Example 2, of the granular occurrence of the eGFP-due green fluorescence (Probe) in cells, by means of fluorescent probe and transmitted light.

FIG. 4-B is a representation of the results of observation, in Example 2, of the appearance after carrying out the intracellular introduction method of the present invention, by means of fluorescent probe and transmitted light.

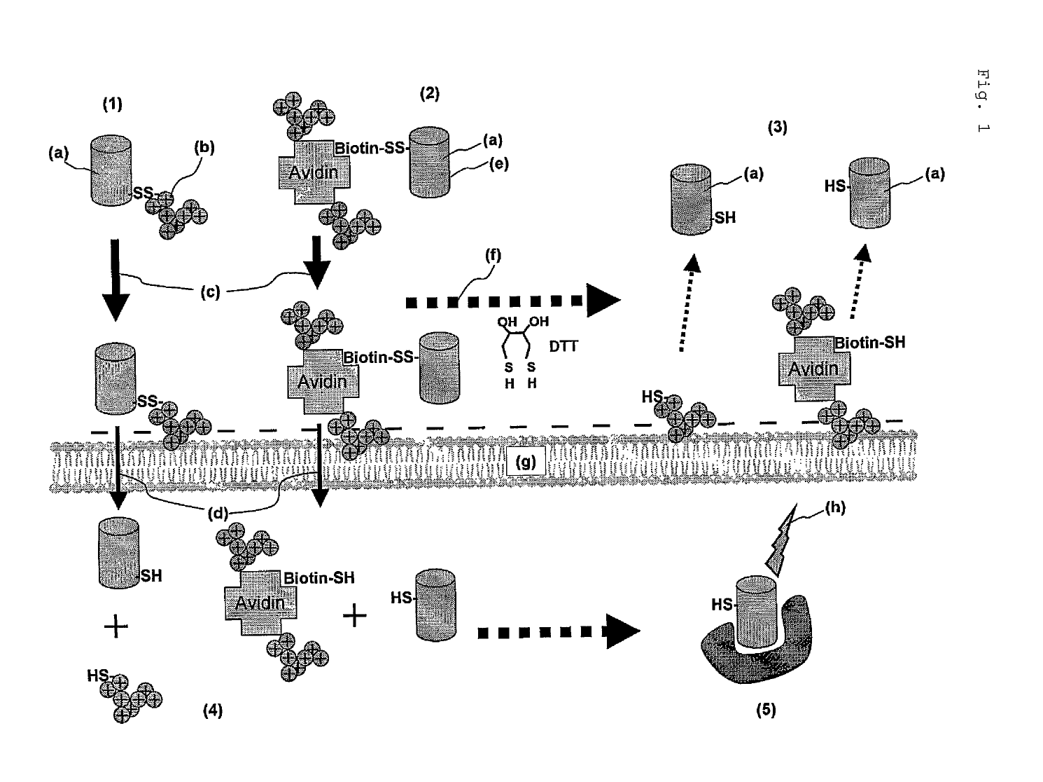

Here are the marks in the drawings.

(1): Cationic polymer-mediated intracellular introduction of a protein

(2): Cationic carrier-mediated intracellular introduction of a protein

(3): Rinsing away/eradication of the fluorescent probe adsorbed on the cell surface

(4): SS bond reduction in an intracellular reductive environment

(5): Binding of the fluorescent probe to an intracellular target molecule and visualization thereof

(a): Protein

(b): Cationic polymer

(c): Adsorption by an electrostatic interaction

(d): Intracellular introduction by endocytosis and/or direct membrane permeation

(e): Fluorescent probe for recognizing a specific molecule

(f): Cell surface treatment with a reducing agent such as DTT under mild conditions

(g): Living cell surface charged negatively

(h): Fluorescent probe

(i): PEI600

(j): eGFP

(k): NLS

(l): Z-axis

### BEST MODE FOR CARRYING OUT THE INVENTION

The following examples illustrate the present invention more specifically. They are, however, by no means limitative of the scope of the present invention. In the examples, “%” represents “% by mass”, unless otherwise specified.

### Example 1

### Preparation of a Fluorescent Probe and a Cationic Carrier for Intracellular Introduction

eGFP-NLS, a recombinant protein resulting from addition of 3 repetitions of the SV40 virus large T antigen-derived nuclear localization signal (NLS) to the carboxyl terminus of the green fluorescent protein (eGFP), was prepared in a form dissolved in phosphate-buffered saline (PBS), pH 7.4, by expression in Escherichia coli, followed by purification. Then, a biotinylating reagent containing an SS bond in a spacer, namely Sulfo-NHS-SS-Biotin (Produced by Pierce), was dissolved beforehand in dimethyl sulfoxide and mixed with the eGFP-NLS in an eGFP-NLS-to-Sulfo-NHS-SS-Biotin mole ratio of 1:4, and the reaction was allowed to proceed at 25° C. for 4 hours. This reaction mixture was purified using a Sephadex G-25 column equilibrated with PBS to give eGFP-NLS-SS-Biotin.

Chicken avidin (1 mg; Produced by Wako Pure Chemical) was dissolved in 0.5 mL of pure water and, then, 0.5 mL of a 20% aqueous solution of polyethylenimine (PEI, average molecular weight 600) as adjusted beforehand to pH 5 with hydrochloric acid was added. Then, a solid water-soluble carbodiimide (EDC) was dissolved in pure water to a concentration of 10 mg/mL and 10 μL of the solution was quickly added to the aqueous avidin/PEI solution (final concentration: 0.5 mg/mL avidin, 10% PEI600-HCl, pH 5.0, 0.1 mg/mL EDC). After allowing the reaction to proceed at 25° C. for 4 hours, the reaction product was purified using a Sephadex G-25 column equilibrated with PBS to give PEI600-Avidin. Further, PEI600-Avidin-RITC was prepared by subjecting avidin-RITC, avidin labeled in advance with rhodamine B (RITC, Produced by Sigma) prior to the PEI coupling reaction, to coupling to PEI600 following the same reaction procedure.

Intracellular Introduction of eGFP-NLS-SS-Biotin

In 100 μL of DMEM medium (serum-free), 1 μM each of the PEI600-Avidin (or PEI600-Avidin-RITC) and the eGFP-NLS-SS-Biotin were mixed together, and the mixture was allowed to stand at room temperature for 5 minutes for the formation of a biotin-avidin conjugate, which was then diluted with 2 mL of DMEM medium (serum-free) (final conjugate concentration 50 nM). As for the cells used in the experiment, mouse fibroblasts (Balb/c3T3) were cultivated overnight on cover glasses (diameter 18 mm) using DMEM medium containing 10% FBS for growth and adhesion. DMEM medium containing 0.5 mL of the above conjugate was added to each cover glass carrying cells adhering thereto, followed by 2 hours incubation in a carbon dioxide gas incubator at 37° C.

Cell Surface Reduction and Rinsing with Dithiothreitol (DTT)

After intracellular introduction of eGFP-NLS-SS-Biotin+PEI600-Avidin by the above technique, the Supernatant was removed, DMEM medium (serum-free) containing 20 mM DTT was added and, after 10 minutes of treatment at 37° C., the medium was replaced again with DMEM medium (serum-free).

### Fluorescent Observation of Cells

After the above introduction procedure, the cells were rinsed once with DMEM medium (serum-free), each cover glass, with the cell-carrying face facing the inside, was sealed in DMEM medium (serum-free) on a slide glass with silicone tape (3M Scotch No. 70) sections attached thereto as spacers, and the cell surface and inside were observed to measure the fluorescence using a confocal laser microscope (Zeiss 510META), followed by data analysis.

### Specific Effects Produced by the Above Means

For demonstrating the present invention, use was made of the observation of the eGFP-due fluorescence, and of eGFP-NLS expected to be transferred to the cell nucleus after intracellular introduction as a result of addition of NLS as the “fluorescent Probe” and PEI600-Avidinor PEI600-Avidin-RITC serving as the carrier for intracellular probe introduction as the “cationic Carrier”. Since it is a key in the practice of the present invention to dispose a solvent-exposed SS bond, a biotinylating reagent containing the SS bond within a spacer (Sulfo-NHS-SS-Biotin) was used.

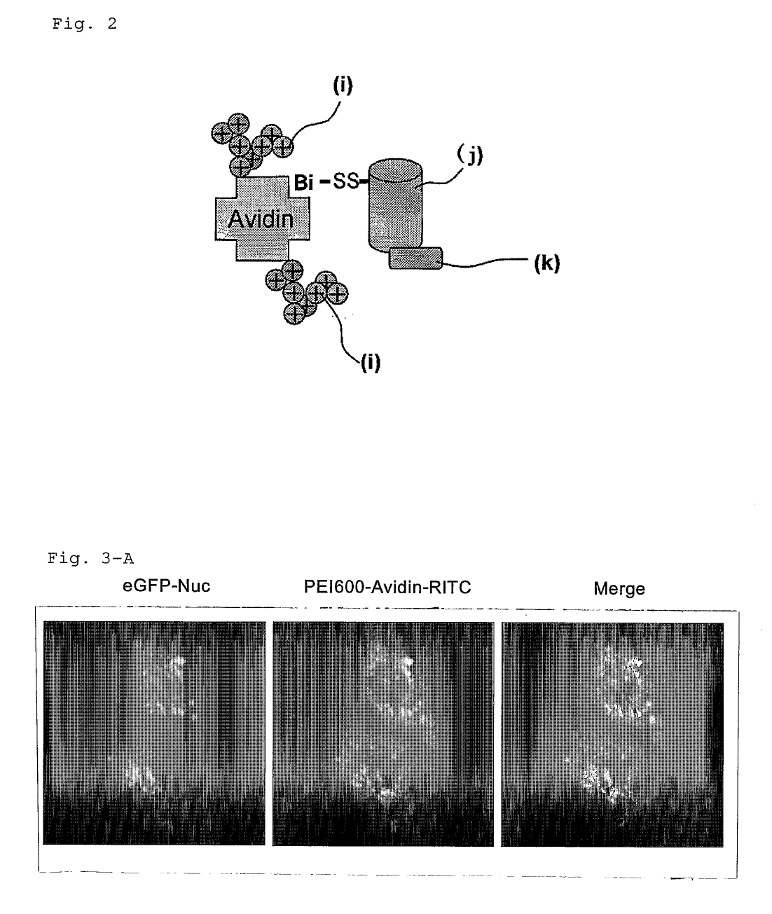

The mode of binding of eGFP-NLS-SS-Biotin and PEI600-Avidin on that occasion is schematically shown in FIG. 2. In FIG. 2, one biotin molecule (Bi) and one disulfide bond (SS) are shown for one avidin molecule, for instance. However, this figure is given only schematically and never indicates that the manner of binding is limited to this mode.

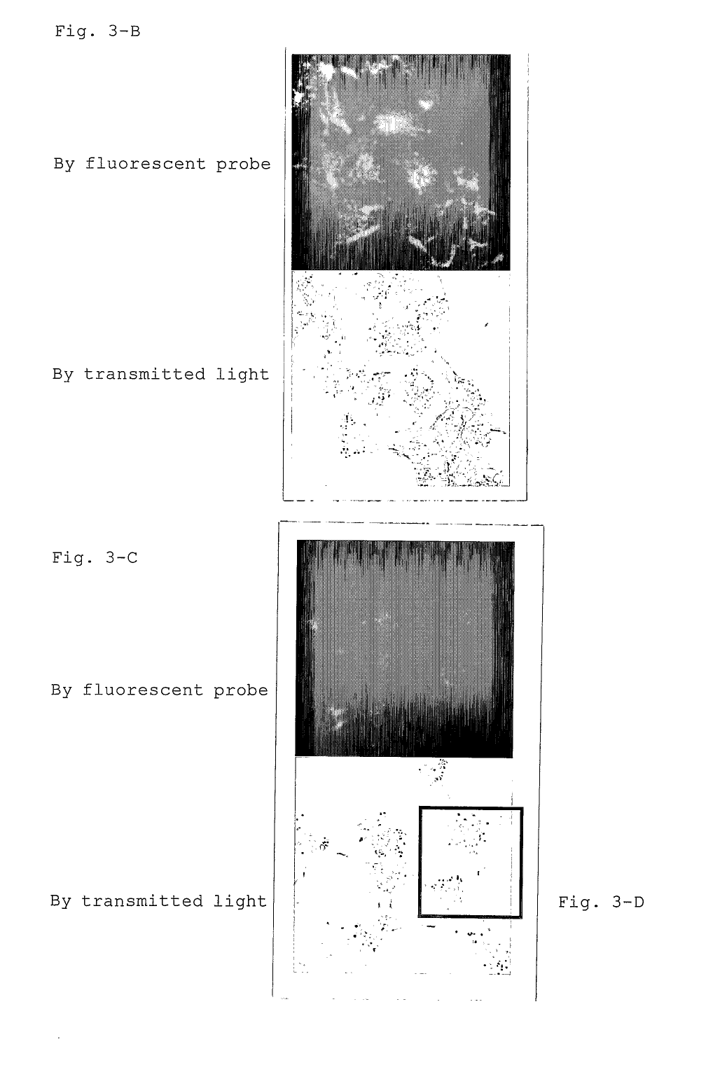

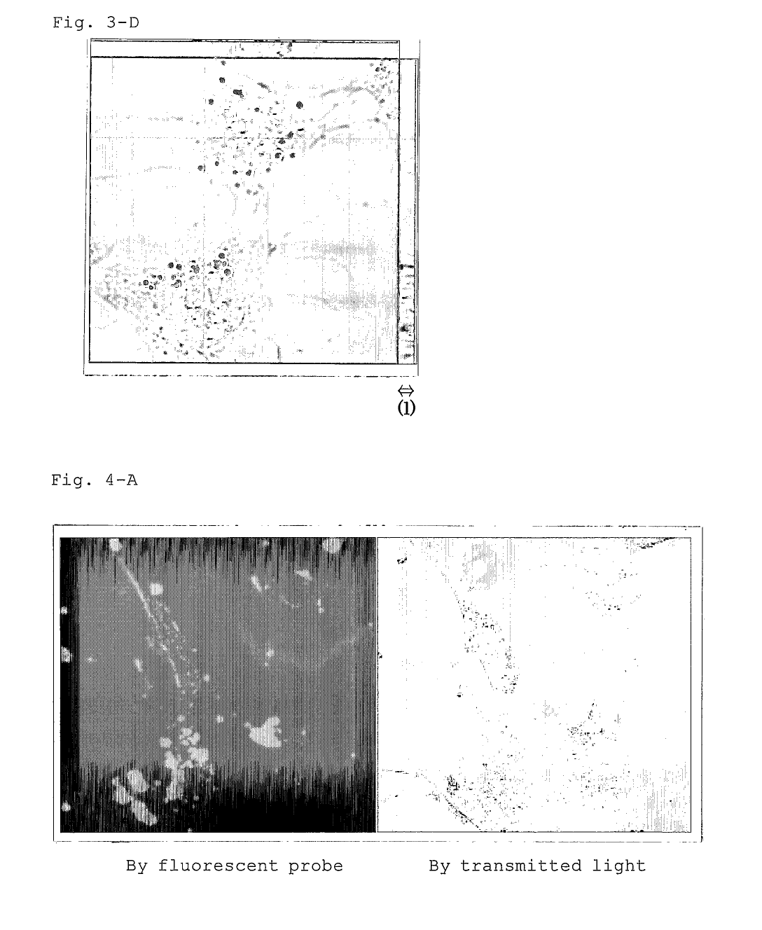

As shown in FIG. 3-A, it was confirmed that the eGFP-due green fluorescence (Probe) and the RITC-labeled PEI-Avidin-due red fluorescence (Carrier) dissociated in cells and the Probe was localized in the intracellular nucleus and the vicinity thereof. In view of these results, the technology can be expected to enable the binding of a probe introduced by this technique to an intracellular target site without any carrier-due restriction in cells.

FIG. 3-B shows the appearance of the cell surface and of the cell inside upon total visualization of the Probe-due fluorescence following intracellular introduction using this technique. On the contrary, it was confirmed in FIG. 3-C that the fluorescence due to the Probe on the cell surface could be rinsed off and erased rapidly by mere 10 minutes of treatment of the cell surface with DMEM medium containing 20 mM DTT just before observation. No influences on living cells were observed under these treatment conditions, indicating that this technology was accomplished under very mild conditions.

FIG. 3-D shows the results of further analysis, after enlargement of a part of the image observed in FIG. 3-C, of the localization of the intracellular fluorescence in the direction of the Z-axis. These results demonstrated that the eGFP-NLS introduced into cells can be transferred to the intracellular nucleus in a reliable manner and that low-background analysis is now possible as a result of the disappearance of the cell surface fluorescence.

### Example 2

### Preparation of a Fluorescent Probe and a Cationic Carrier for Intracellular Introduction

A biotinylating reagent containing an SS bond in a spacer: Sulfo-NHS-SS-Biotin (Produced by Pierce) was dissolved in dimethyl sulfoxide in advance and eGFP and Sulfo-NHS-SS-Biotin were mixed together in a molar mixing ratio of 1:4 and the reaction was allowed to proceed at 25° C. for 4 hours. This reaction mixture was purified using a Sephadex G-25 column equilibrated with PBS to give eGFP-SS-Biotin.

Chicken avidin (1 mg; Produced by Wako Pure Chemical) was dissolved in 0.5 mL of pure water, and 0.5 mL of a 20% aqueous solution of polyethylenimine (PEI; average molecular weight 600) as adjusted beforehand to pH 5 with hydrochloric acid was added thereto. Then, a water-soluble solid carbodiimide (EDC) was dissolved in pure water to a concentration of 10 mg/mL, and 10 μL of the solution was quickly added to the aqueous avidin/PEI solution (final concentrations: 0.5 mg/mL avidin, 10% PEI600-HCl, pH 5.0), 0.1 mg/mL EDC). The reaction was allowed to proceed at 25° C. for 4 hours, and the reaction mixture was purified using a Sephadex G-25 column equilibrated with PBS to give PEI 600-avidin.

### Synthesis of a Carboxyl Group-Containing Polymer

A carboxyl group-containing polymer showing a pH-dependent solubility feature was synthesized as an endosome-disrupting substance.

Styrene (55 weight parts) and 70 weight parts of monoisopropyl maleate were subjected to suspension polymerization in water in the conventional manner to give a carboxyl group-containing polymer. The pH-dependent solubility feature of this polymer was checked and it was confirmed that a 1% aqueous solution undergoes insolubilization at pH 6.5 (25° C.)

Intracellular Introduction of eGFP-SS-Biotin

A biotin-avidin conjugate was prepared in advance by mixing up 1 μM each of PEI600-Avidin and eGFP-SS-biotin in 100 μL of DMEM medium (serum-free) and allowing the mixture to stand at room temperature for 5 minutes. The reaction mixture was diluted with 2 mL of DMEM medium (serum-free) (final conjugate concentration 50 nM). As for the cells to be used in the experiment, mouse fibroblasts (Balb/c3T3) were cultured overnight on cover glasses (18 mm in diameter) using DMEM medium containing 10% FBS for growth and adhesion. DMEM medium containing 0.5 mL of the conjugate solution was added to each cover glass carrying living cells adhering thereto, and cultivation was carried out at 37° C. for 2 hours in a carbon dioxide incubator.

Treatment of Intracellular Endosomes with the Carboxyl Group-Containing Polymer

After intracellular introduction of eGFP-SS-Biotin+PEI600-Avidin by the above technique, the supernatant was removed, DMEM medium (serum-free) containing 50 μg/mL of the carboxyl group-containing polymer was added and, after 2 hours of treatment at 37° C., the cells were returned to DMEM medium (serum-free).

Cell Surface Reduction and Rinsing with Dithiothreitol (DTT)

After the above treatment, the supernatant was removed, DMEM medium (serum-free) containing 20 mM DTT was added and, after 10 minutes of treatment at 37° C., the cells were returned to DMEM medium (serum-free).

### Fluorescence Observation of Cells

The cells subjected to surface reduction and rinsing with dithiothreitol (DTT) and the cells not treated were respectively rinsed once with DMEM medium (serum-free), each cover glass, with the cell-carrying face facing the inside, was sealed in DMEM medium (serum-free) on a slide glass with silicone tape (3M Scotch No. 70) sections attached thereto as spacers, and the cell surface and inside were observed to measure the fluorescence using a confocal laser microscope (Zeiss 510META), followed by data analysis.

### Specific Effects Produced by the Above Means

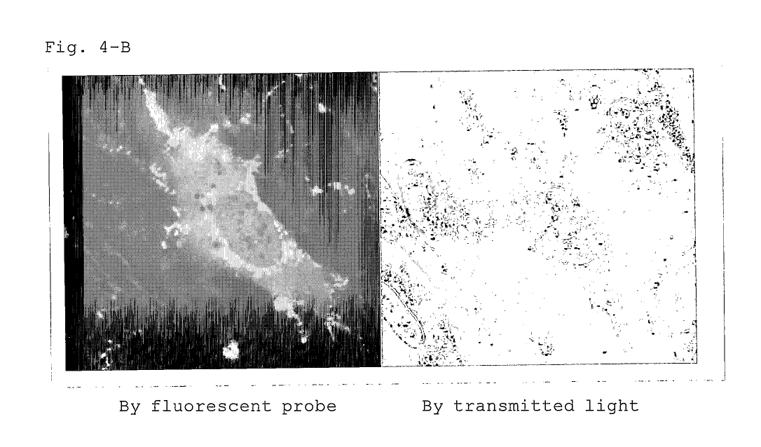

As shown in FIG. 4-A, the eGFP-due green fluorescence (Probe) occurred like granules within cells. On the other hand, the fluorescence was found uniformly within the cells treated with the carboxyl group-containing polymer and eGFP was found diffused uniformly from endosomes into the cytoplasm, as shown in FIG. 4-B. It was thus revealed that the background light retained in endosomes could be reduced.

The present application claims priority under 35 U.S.C. §119 to Japanese Patent Application No. 2004-151853 filed May 21, 2004, entitled “METHOD FOR INTRODUCING PROTEIN AND/OR PEPTIDE INTO CELL”. The contents of that application are incorporated herein by reference in their entirely.

1. A method for introducing a protein and/or peptide into a cell,
which comprises the process of introducing a conjugate generated by binding of a compound adsorbable on a cell surface to a protein and/or peptide via a polysulfide bond into a cell, and treating with a reducing agent.

2. A method for introducing a protein and/or peptide into a cell,
wherein the protein and/or peptide is a labeled one,
the method comprising the process of adding an endosome-disrupting substance in a living cell to thereby disrupt the endosome.
